(19) United States
(12) Patent Application Publication
(10) Pub. No.: US 2007/0173704 A1
(43) Pub. Date: Jul. 26, 2007

(54) Corporeal condition assessment and food selection

(76) Inventors: Irina Bjorno, Taastrup (DK); Thomas H. Neighbors, Vienna, VA (US); Leif Bjorno, Taastrup (DK)

Correspondence Address: Thomas H. Neighbors III, 419 Cynthia Lane N.E., Vienna, VA, 22180, US

(21) Appl. No.: 11/652,612

(22) Filed: Jan. 12, 2007

Related U.S. Application Data
(60) Provisional application No. 60/762,824, filed on Jan. 26, 2006.

(51) Int. Cl. A61B 5/00

(52) U.S. Cl. 600/300

(57) ABSTRACT

Disclosed are methods and systems for determining a body condition, for food selection by type and/or amount and for establishing a food identification system which may be used therewith.

### CROSS REFERENCE TO RELATED APPLICATIONS

This non-provisional patent application is based on “Corporeal Condition Assessment and Food Selection,” U.S. Provisional Patent Application No. 60/762,824, dated 26 Jan. 2006.

### FEDERALLY SPONSORED RESEARCH OR DEVELOPMENT

NONE

### FIELD OF DISCLOSURE

Disclosed here are methods and/or systems for measuring and/or monitoring the health and/or physical condition of the corporeal body and/or for determining an appropriate diet or preferred physical nourishment based thereon.

### BACKGROUND

The body of any living organism is like any machine. The better the service and the better the fuel, the better both the machine and the body will function. In short, the corporeal body, whether human or of any other living being, cannot perform better than the food or nourishment or fuel provided thereto permits. Provision of the proper food or nourishment types to the body at the appropriate times may be understood to yield the best functional situation and best health for the body. Of course, contrarily, provision of the wrong food types to the body may typically produce bodily problems which in some instances may result in the creation of overweight issues or worse, degradation into illness or disease.

More problematically, the choices of what foods may be most appropriate to eat, particularly at what corresponding times has not been well-described. For the most part, suggestions in the art of what and how much to eat have generally failed to take into account the variable state of the body at any distinct time. The current physical state or condition of the human body is not often or easily regarded in determining which of various food choices may provide the better resulting effect. Still furthermore, it appears that the state of the art has not definitively determined which body parameters would or should provide the best insights into which food selections might best be made at any particular time and any particular conditions of the body.

### SUMMARY

Disclosed here are methods and/or systems directed toward optimizing food or nourishment selection for a corporeal body based on a type of “energy” monitoring of the body. The goal is to provide food selections which assist in moving the body to what will here be deemed an optimal neutral or balanced “energy” state. The contrasting alternative non-balanced “energy” states here are defined as alternately either low or high “energy” states. For achieving this goal, a solution is to take into account the body's current state, whether high, low or neutral, so as to obtain or maintain an optimal neutral “energy” state. Particular foods can then be selected corresponding to either the low, high or neutral state. Alternatively, the amount of food may be calculated to achieve a standard normal weight.

A first method hereof may include a determination of the physical condition or state of the body. In one instance, this may be accomplished by measuring the pulse (the number of heart beats per minute) and the respiration (the number of breathing cycles per minute); and then forming a ratio thereof, pulse to respiration, to define the “condition” of the body. In one of a variety of embodiments, these parameters may be determined for the body at rest. Also, in some embodiments, particular values may be calculated and used. For one example, three alternative ratios may be obtained; namely, values less than about 5 which may represent low “energy” states, values greater than about 6 representing high “energy” states and values between about 5-6 which are here defined as optimal or balanced, neutral “energy” states.

A further method may involve the determination and/or selection of a particular food, by type and/or amount, corresponding to the determined physical state or condition. Foods suitable to correct low “energy” states may be those which easily and/or quickly generate energy or heat to counter the low “energy” state, whereas, slow energy converting foods may be chosen for ingestion during high “energy” conditions. A third group of foods may present a relatively balanced “energy” profile for maintenance of a neutral energy state.

Implementation of a selection process hereof may include a color or other identification or coding scheme which may involve the pre-imposition of distinctive labels on packages associated with particular food types to notify the user of the particular grouping to which the particular food belongs so that the user can appropriately select foods corresponding to their discrete periodic state or condition. The identification system can be used for assistance in food selection whether at home or from a menu at a restaurant, or in grocery selection at the market. In any of these, the coding can further provide for organization of the grocery market, the menu or even the home refrigerator or pantry, wherein the coding can provide for grouping like foods together for ease of selection.

### BRIEF DESCRIPTION OF THE DRAWINGS

FIG. 1 is a flow diagram for a process according to the disclosure hereof.

FIG. 2 is a further flow diagram for another process according to the disclosure hereof.

### DETAILED DESCRIPTION

The disclosure hereof is directed toward the optimizing of the functioning and/or health of a corporeal body through food selection by type and/or amount which may be related to a type of “energy” measurement and/or monitoring of the body. A goal may be to provide food selections by type and/or amount which may assist in moving the body to what will here be deemed an optimal neutral or balanced “energy” state. The contrary, non-balanced alternative “energy” states may be here defined as alternately either low or high “energy” states, such as respective low activity/cold or high activity/warm activity states. A solution hereof is to provide food selections which take into account the currently experienced state, high, low or neutral, so as to drive the body to or maintain the body in an optimal neutral activity or balanced “energy” state. Thus, food intake in a low “energy” state situation would preferably be of a higher energy-producing food type. Conversely, for a body in a high “energy” state, the consequent food intake would preferably be of a type which offsets the high “energy” level. A neutral, or balanced “energy” state herein is one which is neither too low, nor too high.

Note, the term “energy,” or it's sometime alternative, “activity”; whether high, low or neutral, when related to the body state or condition herein is not strictly related to the scientific concept of “energy”, which is a generally non-negative phenomenon. Rather, the “energy” hereof when used in relation to the body state or condition is an assessment of the relative vigour, liveliness or physical excitation or agitation as opposed to any lethargy, sluggishness, coldness or other lower body activity subjectively and effectively borne or experienced by the particular corporeal body in question. It may thus take into account such relations as the overall amount of food or nourishment available within the body, and/or the body's ability to utilize or convert such food or nourishment, as well as what types of food or nourishment is provided thereto.

A first process hereof may include a determination of the instantly-occurring physical condition or state of the body at a particular time; also referred to as a transient condition herein due to its variable nature. In one implementation, this determination may be achieved by measuring the pulse of the body, typically at rest (e.g., the number of heart beats or strokes per minute) and the respiration of the body, also typically at rest (e.g., the number of complete breathing cycles per minute); and then forming a ratio thereof, pulse to respiration, to thereby define the “condition” of the body. In other words, the “condition” may be defined as equal to (=) the pulse strokes divided by (/) the respiration cycles (or condition=pulse/respiration). In some implementations hereof, three alternative conditions may thus be determined; namely, values of less than about 5 may be deemed to represent low energy states, values greater than about 6 represent high energy states and values about 5 to about 6 are defined as the optimal neutral energy balanced state. In a further alternative implementation, two further intermediate states, e.g., a semi-high or semi-hot state and/or a semi-low or semi-cold state may also be tracked. This could occur in a fashion such that values above a ratio such as 7 may be high, values between about 7 and about 6 are semi-high, values between 5 and 6 being neutral, values between about 5 and about 4 being semi-low, and values below about 4 being low, for one example.

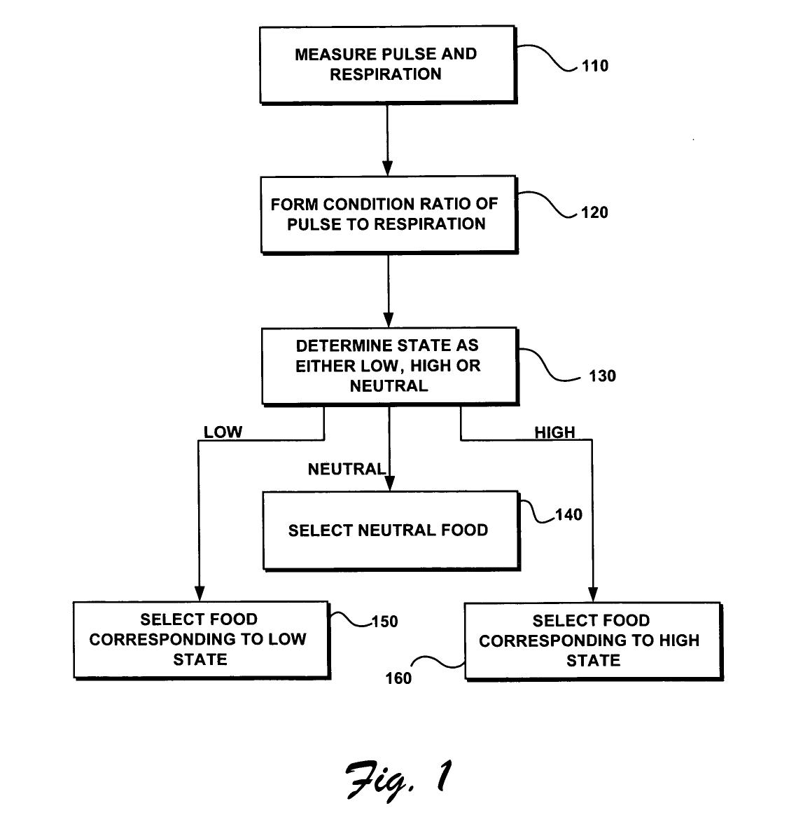

FIG. 1 presents an exemplar illustration of an implementation of this method of determining a physical condition including the respective initial operations 110, 120 and 130. These operations more specifically include the measuring certain physical parameters, per operation 110, here the parameters being pulse and respiration, then the use, per operation 120, of these parameters in forming a ratio. A determination 130 is then made as to the state or condition as either high, low or neutral as described above, or high, semi-high, low, semi-low or neutral.

A further step or operation in an overall health improvement methodology may then be the determination of or selection of a particular food type corresponding to the determined physical state or condition. This is shown in FIG. 1 by the three alternative operations 140, 150, and 160, which are the respective selections of a food type corresponding to the neutral (140), low (150), or high (160) body “energy” state.

If the body is in the low energy state more energy in the food should come from fat and/or fat/proteins. If the body is in the high energy state more of the energy in the food should in general come from complex carbohydrates like juice, fruits and vegetables. In a balanced energy state in the body it could be suggested that about 40% of the energy in the food should come from carbohydrates, 30% from proteins and the remaining 30% from fat. This is only an indication and the valid ratios for a specific person can vary.

Some examples of foods which may be classified in this fashion include the following. For use with high energy body states, sugars and carbohydrates may be selected from foods like many vegetables, fruits, soft drinks and milk products (particularly bovine dairy products). For low energy body states, higher protein/fat-based foods such as fish, chicken, pork, beef and other meats may be selected. “Neutral” foods for the neutral body state may include in some examples, some grain products, corn and corn products.

More examples of food classifications which may prove useful here may include not only the fast-acting, quickly-convertible carbohydrate versus protein distinction. Rather also, some fruits and vegetables may either have complex carbohydrate structures, or low carbohydrate contents with rather high fiber or alternative constituents. These alternative structures may provide benefit to a body in a high energy state, as where the body's digestive system may require redirected energy from the body as a whole for processing. Potatoes and lettuce may provide such advantages. Similarly, other food constituents may also contribute to the food classification as useful for either high or low states. An example here may be L-tryptophan, an amino acid occurring naturally in many foods such as turkey, tuna, milk and peanuts, which may provide a leavening effect, even promoting sleep, to thereby reverse or alleviate a high energy state. A similar contrary example may be caffeine, a naturally-occurring substance in many foods such as coffee, cocoa and chocolate which may provide a wakening effect in the user which may correspond to raising the energy level from a lower energy level state. Other examples may come from known oriental or other food cultures which recognize that some foods are neutral for the body, where other foods provide a “heating-up” of the body to a higher energy level, and others provide a sort of “cooling-down” of the body to a lower energy level.

Implementation of a selection process hereof may include a color or other identification scheme which may involve the pre-imposition of distinctive labels such as color-coded indicia on packages or other labels associated with particular foods to notify the user of the particular grouping to which the particular food belongs so that the user can appropriately select which foods to ingest depending upon their particular state or condition. Such a marking system provides assistance so that the user will not have to remember the classification of any particular food item. The color or other coding system can be used for assistance in food selection whether at home or from a menu at a restaurant, or in grocery selection at the market. In any of these, the coding can further provide for organization of the grocery market, the menu or even the home refrigerator or pantry, wherein the coding can provide for grouping like foods together for ease of user selection.

In one exemplar implementation, the foods may be classified by color marks, as for example using respective colors like green, red and blue. Here, the foods may be classified by marking for example, neutral foods with a green color mark, and foods for use in providing higher energy (as by “heating-up” the body to a higher energy level) with a red color, and foods for providing a semblance of a lower energy (as by “cooling-down” the body to a lower energy level) with a blue color.

In use, it may be determined to mark only some types or sub-types or perhaps to mark all types of food in this way. Foods thus marked (e.g., with a color-coding or other indicia) may then be easily identified by a user when shopping in a grocery store, or when dining in a restaurant, as when the dishes are marked on the menu. The user then does not have to remember the color or classification of all the food items, i.e., whether they are red, green or blue.

A further implementation may involve the grouping together of different food items of the same classification. Here, the store, shop, restaurant menu or home storage may be divided by the different food types/classifications. For example, the store may be sub-divided by areas within the store representing the classifications of food types (e.g., the red, green and blue foods) where the types of food are grouped together. Thus, three discrete areas of the store may be dedicated to the respective food groups (e.g., one for red food items, another for green food items and the third for blue food items). Similarly, the storage of food in a home may also be made to follow this system. In one example, the refrigerator may be separated into three domains; one for each of the types of food (e.g., one for red food items, one for green food items and one for blue food items). Indeed, the refrigerator may have the subdivisions built therein, to provide the highest simplicity for use by the end consumer.

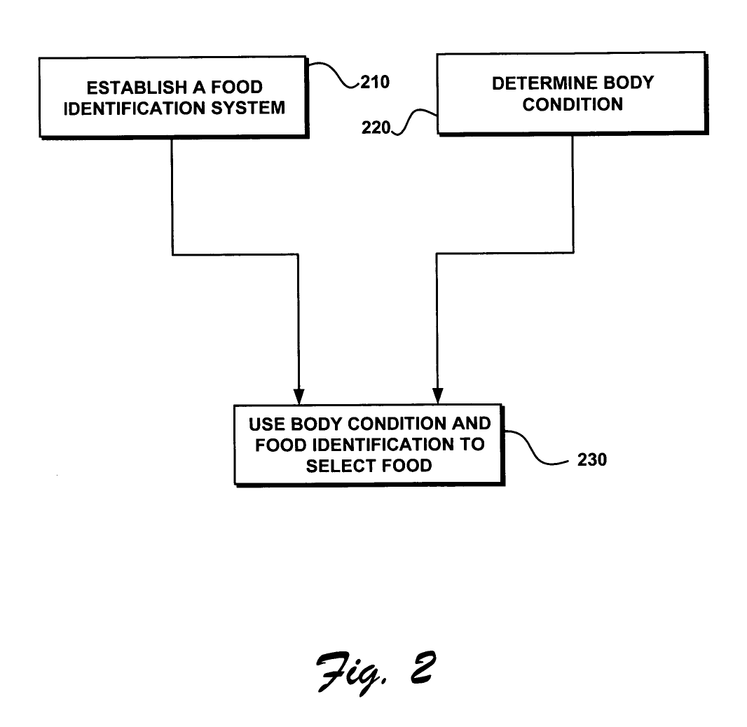

FIG. 2 provides an illustration of the use of a marking system in an overall methodology according hereto. For example, an operation 210 of establishing an identification system may be used with an operation 220 of determining a body condition together in a culminating operation 230 for selecting a particular food. Note, the operations 210 and 220 need not take place in any order, either may occur before the other, or they may take place substantially simultaneously.

In a further alternative implementation, whether in addition to or in lieu of the above concepts, the weight or amount of the food may be calculated for the individual to intake for their particular condition. A goal is to calculate the overall caloric balance, using in some examples 2000 Kcal per day, so that the system also can take account for the total energy intake for weight control purposes. In some implementations hereof, a calculation may be adapted to not only provide options for food selection, but may also provide a preferred or otherwise desirable amount, e.g., by weight or other measure, for the individual to eat to aim for an appropriate or otherwise standard daily intake of calories, or other nutritional value, as for example, a carbohydrate, protein, fat, vitamin or other measure. Such a provided intake amount may depend on the individual's age, weight, health condition, or the like.

Thus disclosed are a variety of methods and/or methodologies for either or both determining a body condition and selecting a food appropriate to the condition. Color or other indicia marking systems provide assistance here as well. Moreover, other assistance systems or devices may also be implemented. A first example includes one or more measuring devices for the measuring of the body parameters of pulse and/or respiration. These may be separate and/or manually operable devices or they may be connected to each other and/or to a device for forming the ratio. In other words, the pulse and respiration devices may either be manually operable for the user to form the ratio, or the pulse and respiration devices may output their measurements to one or the other of each other, or to a third device, any of which being enabled with calculation ability to form the ratio of pulse to respiration and output that result (as by a display). Indeed, one or more of these devices may also include a display which displays to the user either the ratio result or the color-code corresponding to the result so that the user need only know the color-code for food selection. For example, if the ratio provides a lower than 5 value, thus being a low “energy” condition, the display may either or both provide the number or merely a color, such as the red color in the above examples, so that the user knows to select foods from the red group.

In an implementation, a measuring device may be provided which can measure synchronically both of the parameters pulse and respiration cycles. Similarly, one or the other or both of the measuring devices may also be adapted for chronologically measuring the input parameters; i.e., the pulse and respiration cycles. This may provide for regular monitoring of the body condition. Such a chronological monitoring may be graphical presented, and may include the parameters themselves and/or may include the resulting ratio parameter or “condition”, which may be done in the color-coded system, e.g., with a red, green and blue color in the background to indicate the energy state of the body.

Any or all of these devices may further include memory for tracking either or both of the parameters (pulse and/or respiration) and/or the resulting ratio for longer term monitoring of the user's food intake needs. Indeed, a computing device or system and/or software may be utilized in the calculations and/or tracking, and/or in further predicting proper diet based upon these inputs. A software product may also be provided which may utilize the measured values of pulse and respiration cycles to view progress over time and to provide suggestions for the daily diet; as for example where trends are spotted, and/or where alterations may be made.

Thus, one or more devices hereof may have one or more functionalities. Indeed, in many implementations, combined measuring functionalities (for the two primary parameters, pulse and respiration) may be provided in combination with hardware and/or software products in a combined functionality system. Moreover, such a system may either be provided on a hand-held computing device, or be disposed on a conventional computing system which is adapted to be linked to a hand-held device. Still further, such a system may be linkable to a medical system for communication to or with a medical doctor, or in similar fashion to another system for communication of data (inputs or results or both) to or with a non-medical person, such as a dietician or personal trainer any of whom may be engaged to provide recommendations on the dieting plan.

Note, the computer connections may be by option of a direct link between computing devices (PC or other devices, like a hand-held computer); or other connections, e.g., wireless, may be had. Moreover, the software functionality may be implemented on or through connection to the local or wide area network connections or to or through the internet (also known as the world-wide web) or partly on the internet and/or other connections. Such connections can provide for the transfer of pertinent data to a medical doctor or another person who as a response provides suggestions for the diet plan.

Note, as introduced above, the system hereof may include a data processing means, which can be any type of general-purpose computer, such as a personal computer (PC), workstation, handheld computer, electronic personal assistant or mobile phone. The operating system may be any kind of commercially available software package, such as Linux, Windows (XP, NT, ME, CE), Mac OS, PalmOS, EPOC, etc. The system may further include a means for storing parameter values, such as a hard disc, flash memory or any other memory. The system may typically further include a data input interface, such as a keyboard, keypad, touch screen, or mouse, and a data output interface such as a computer screen, television screen, or any other display. The systems/methodologies may be herein described as including a PC as the data processing means, keyboard and mouse as the data input interface, and computer screen as the data output interface.

Consequently, the methods and/or systems hereof may be implemented in or on a computer and/or may be provided as a software product, which uses the measured values of pulse and respiration cycles to predict and suggest the user's diet. As a computer or system input, the two measured parameters pulse and respiration cycles may be used as described above, and, moreover, the chronological development of the parameters, as well as the resulting condition parameter may be monitored, and may be shown graphically, inter alia.

The energy states may be tracked for purposes of food selection or otherwise for general health concerns. Thus, herein, the respective low and/or high “energy” states may include or be alternatively directed to situations such as the following. In an example, the low state may include a situation where the body is slowing down, a situation which may include symptoms such as cold feet or other extremities. Thus, health issues of minor cold and/or slowing down may be determined, or more drastically, issues such as hypothermia may be monitored. On the other hand, the high state may include or be representative of energetic situations other than mere high blood sugar level in the body; and, may thus also include agitation, excitation, or other feelings of being troubled, restless or stressed. For these and other health determinations, the neutral or balanced energy situation may be an optimal target for the well-being of the user and may assist in issues of or attempts at weight loss, weight gain, feeling well, happy and/or relaxed. Furthermore, as an option, one or more of the problems like sleeping disorders, stress, cold, osteoporosis, diabetes, high blood pressure, heart diseases, cholesterol, digestive problems, being overweight or underweight, can be tracked and/or indicated as well; and a remedy or remedies may be incorporated into the diet plan. Target body weights may be made by tracking or providing a desirable weight or amount of the food for individual intake. A computer program may provide options for food selection, and corresponding amounts thereof to eat to aim for a standard daily intake of calories, or carbohydrates or other measure. This intake amount may depend on the person's age, weight, health condition, etc.

In some implementations, one or more software functionalities may be provided. For example, a first software functionality may be based on the desired balance between carbohydrates, proteins and fat in the food. In this or a further alternative hereof, the software functionality may also/alternatively be based on a desired total food energy intake per day (in some examples using 2000 Kcal per day), and thereby calculates the amount of food constituents in the meals: e.g., the relative amounts of fruits, vegetables, meat, bread, etc. to enable the user to obtain the desired balance between carbohydrates, proteins and fat in the food and at the same time have the desired total energy intake per day. An alternative software functionality may be based on the desired balance between carbohydrates, protein and fat, and desired energy intake per day (for example 2000 Kcal per day), and the user's specific wishes of eating specific food products, like cake, fruits, juices, fish, bread, etc., and this example, the software calculates how much the user can eat of each desired item, though it would also eventually likely be desired to have the software also calculate suggestions for intake of supplementary food items in order to continue to provide a desired balance between carbohydrates, proteins and fat. A further alternative software functionality may be based on the results of either of these alternatives and may further be established to calculate how much of any one or more different vitamins and minerals there are in the food and compare these figures with the recommended daily doses to determine whether it may be desirable to intake supplementary vitamins, as for example vitamin tablets.

Although the present invention has been described in detail for purpose of illustration, it is understood that such detail is solely for the purpose, and variations can be made therein by those skilled in the art without departing from the scope of the invention. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

What is claimed is:

1. A method for determining a physical condition of a living body comprising:
measuring a pulse and a respiration rate of a living human body; the pulse being the number of heart strokes per time period and the respiration being the number of breathing cycles per time period; and,
forming a ratio of a physical condition which is the pulse divided by the respiration; and,
determining the physical condition as being one of three alternatives; namely, low, neutral or high; using the ratio of the physical condition.

2. A method according to claim 1 wherein the low, neutral and high alternatives correspond respectively to the body being in respective low, neutral and high energy states.

3. A method according to claim 2 wherein the neutral energy state is an optimal balanced energy state.

4. A method according to claim 2 wherein the low energy state is representative of the body being in a low energy level state.

5. A method according to claim 2 wherein the high energy state is representative of the body being in a high energy level state.

6. A method according to claim 1 wherein the low alternative is defined by the ratio being lower than about 5, the neutral alternative being defined by the ratio being between about 5 and about 6, and the high alternative being defined by the ratio being greater than about 6.

7. A method according to claim 1 further including one or more additional states, the one or more additional states including one or both of a semi-high and a semi-low state.

8. A method according to claim 7 wherein the low alternative is defined by the ratio being lower than about 4, the semi-low alternative is defined by the ratio being between about 4 and about 5, the neutral alternative is defined by the ratio being between about 5 and about 6, the semi-high alternative is defined by the ratio being between about 6 and about 7, and the high alternative is defined by the ratio being greater than about 7.

9. A method according to claim 1 further including selecting one or more of a particular food type or amount corresponding to the determined physical state or condition.

10. A method according to claim 8 wherein one or more of:
the particular food type is a food for a low energy state which generates energy or heat to counter the low energy state;
the particular food type is a food for a high energy state which diverts energy during high energy conditions; and,
the particular food type is a food for a neutral energy state which presents a balanced energy profile for maintenance of a neutral energy state.

11. A method according to claim 9 wherein:
the food for the low energy level provides for countering the low energy level;
the food for the high energy level provides for countering the high energy level; and, the food for the neutral energy level provides for maintaining the neutral energy level.

12. A method according to claim 9 wherein one or more of:
the food suitable for the low energy level is one or more of high protein/fat or high fat or high caffeine or alcohol to provide for countering the low energy level;
the food suitable for the high energy level is one or more of high carbohydrates, high fiber, complex carbohydrate or high L-tryptophan to provide for countering the high energy level; and,
the food for the neutral energy level is for example a grain or a corn product to provide for maintaining the neutral energy level.

13. A method according to claim 8 further including selecting an amount of a particular food for achieving a body weight objective.

14. A method according to claim 8 further including:
implementing an identification scheme which further involves;
pre-imposing distinctive labelling on particular foods to notify of particular grouping to which the particular foods belong.

15. A method according to claim 9 to assist in control and adjustment of body weight.

16. A method according to claim 9 to assist in the prevention of life style disorders and minor disorders.

17. A method according to claim 14 wherein the identification scheme includes a color coding system.

18. A method according to claim 17 wherein the color coding system includes use of red, blue and green colors.

19. A method according to claim 18 wherein:
the red color is used for foods corresponding to the low energy level;
the blue color is used for foods corresponding to the high energy level; and,
the green color is used for foods corresponding to the neutral energy level.

20. A method according to claim 19 wherein:
the red color foods provide for countering the low energy level;
the blue color foods provide for countering the high energy level; and,
the green color foods provide for maintaining the neutral energy level.

21. A method according to claim 19 wherein one or more of:
the red color foods are one or more of Protein/fat or high fat or high caffeine to provide for countering the low energy level;
the blue color foods are one or more of high carbohydrates, high fiber, complex carbohydrate or high L-tryptophan to provide for countering the high energy level; and,
the green color foods are for instance a grain or a corn product to provide for maintaining the neutral energy level.

22. A method according to claim 14 wherein the identification system is used in one or more of: a food market; a restaurant; or a home.

23. A method according to claim 22 wherein the identification system provides for one or more of: organization of the grocery market, grouping of items on the menu at the restaurant, or organization of foods in a home refrigerator or pantry.

24. A method according to claim 23 wherein one or more of market, the restaurant, the menu, the home, the pantry and the refrigerator are pre-prepared for organization according to the identification system.

25. A method according to claim 1 which is adapted to provide for optimizing food selection for a living body based on the determined physical condition of the body wherein the food selection assists in moving the body toward an optimal neutral or balanced energy state.

26. A bodily parameter measuring device for use in performance of the method of claim 1.

27. A computer-implemented system including one or more of hardware, software or firmware for performing the method of claim 1.

28. A bodily parameter measuring device for use with the computer-implemented system of claim 27.

29. A bodily parameter measuring device according to claim 28, wherein the measuring device is adapted to measure one or both of pulse and respiration.

30. A bodily parameter measuring device according to claim 29 wherein the measuring device is adapted for one or both of synchronically measuring both of the parameters of pulse and respiration cycles and chronologically measuring pulse and respiration cycles.

31. A software product according to claim 27 utilizing measured values of pulse and respiration cycles to provide one or more of daily diets and time progression analysis.

32. A computer-implemented system according to claim 27 which is adapted to present graphical results for one or more of the pulse, respiration, and the condition ratio.

33. A computer-implemented system according to claim 32 wherein the graphical presentation of the parameter “condition” is provided with a red, green and blue color in the background to indicate the energy state of the body.

34. A computer-implemented system according to claim 27 wherein the computer is connected by one of a direct link, a wireless link, a local area network, a wide area network and the internet.

35. A computer-implemented system according to claim 27 wherein the computer is a Personal Computer (PC) and or a hand-held computing device.

36. A computer-implemented system according to claim 26 wherein the computer is adapted to communicate data over a network to a remote system for one of evaluation, analysis and development of a diet or diet suggestions by a doctor or other remote personnel.

37. A method of optimizing a body condition, the method comprising:
starting with a physical state of the body; and
selecting one or both of a particular food type and/or amount based upon the physical state of the body.

38. A method according to claim 37, wherein starting with a physical state includes:
measuring a pulse and a respiration rate of a living body; the pulse being the number of heart beats per minute and the respiration being the number of complete breathing cycles per minute; and,
forming a ratio of a physical condition which is the pulse divided by the respiration; and,
determining the physical condition as being one of three alternatives; namely, low, neutral or high; using the ratio.

39. A method according to claim 38 wherein the low alternative is defined by the ratio being lower than about 5, the neutral alternative being defined by the ratio being between about 5 and about 6, and the high alternative being defined by the ratio being greater than about 6.

40. A method according to claim 38 further including one or more additional states, the one or more additional states including one or both of a semi-high and a semi-low state.

41. A method according to claim 40 wherein the low alternative is defined by the ratio being lower than about 4, the semi-low alternative is defined by the ratio being between about 4 and about 5, the neutral alternative is defined by the ratio being between about 5 and about 6, the semi-high alternative is defined by the ratio being between about 6 and about 7, and the high alternative being defined by the ratio being greater than about 7.

42. A method for optimizing food/nutrient usage for a physical body, the method comprising:
establishing a food identification system;
determining a body condition; and
using the body condition and the food identification system to select a food for consumption.
